(19) United States
(12) Patent Application Publication
(10) Pub. No.: US 2026/0325017 A1
(43) Pub. Date: Oct. 22, 2026

(54) METHODS, DEVICES AND SYSTEM FOR PROVIDING DIABETIC CONDITION DIAGNOSIS AND THERAPY

(71) Applicant: Abbott Diabetes Care Inc., Alameda, CA (US)

(72) Inventors: Gary Alan HAYTER, Oakland, CA (US); Daniel Milfred BERNSTEIN, El Granada, CA (US); Erwin Satrya BUDIMAN, Fremont, CA (US)

(21) Appl. No.: 19/687,853

(22) Filed: May 26, 2026

Related U.S. Application Data
(63) Continuation of application No. 18/117,699, filed on Mar. 6, 2023, now Pat. No. 12,648,717.
Application No. 18/117,699 is a continuation of application No. 15/926,919, filed on Mar. 20, 2018, now Pat. No. 11,596,330.
(60) Provisional application No. 62/474,605, filed on Mar. 21, 2017.

(51) Int. Cl. A61B 5/145; A61B 5/00; A61B 5/1486

(52) U.S. Cl. CPC A61B 5/14532; A61B 5/0004; A61B 5/1451; A61B 5/1486; A61B 5/4848; A61B 5/4866

(57) ABSTRACT

Methods, devices and system for determining fasting glucose level information and post-prandial glucose level information for diagnosing pre-diabetic and diabetic conditions based on monitored glucose measurements are provided.

### RELATED APPLICATIONS

The present application is a continuation of U.S. patent application Ser. No. 18/117,699, filed Mar. 6, 2023, which is a continuation of U.S. patent application Ser. No. 15/926,919, filed Mar. 20, 2018, now U.S. Pat. No. 11,596,330, which claims priority to U.S. Provisional Application No. 62/474,605, filed Mar. 21, 2017, entitled “Methods, Devices and System for Providing Diabetic Condition Diagnosis and Therapy,” the disclosures of each of which are incorporated herein by reference for all purposes.

### BACKGROUND

The detection and/or monitoring of glucose levels or other analytes, such as lactate, oxygen, A1C, or the like, in certain individuals is vitally important to their health. For example, monitoring of glucose level is particularly important to individuals with diabetes and those with conditions indicative of onset of diabetes, in particular to identify physiological conditions that precede the onset of diabetes. If such physiological conditions can be identified in a timely manner, treatment or therapy by way of medication, exercise regimen, and/or modification to diet can effectively diminish the likelihood of diabetic conditions.

Techniques exist to determine if an individual has diabetes or has conditions indicative of onset of diabetic conditions, but these have drawbacks. Such techniques include fasting glucose test and oral glucose tolerance test. A fasting glucose test requires the individual to refrain from eating (i.e., fast) for a certain number of hours (e.g., 8 hours) before performing a blood glucose measurement. Consistent blood glucose measurement below 100 mg/dL is considered normal, measurements that are between 100 mg/dL and 126 mg/dL are considered to represent pre-diabetic condition, and measurements that are greater than 126 mg/L are considered to indicate diabetic condition. These are guidelines to assist physicians in their assessments of individuals suspected of having onset of diabetes. However, fasting for a prolonged period of time before the blood glucose measurement is performed may be inconvenient or not possible with certain individuals, resulting in potentially undiagnosed pre-diabetic or diabetic conditions.

An oral glucose tolerance test also requires that the individual refrain from eating for a predetermined time period (e.g., 8 hours) before performing a blood glucose measurement. Immediately following the blood glucose measurement after fasting for the predetermined time period, as part of the oral glucose tolerance test, the individual drinks a 75 gram oral dose of glucose solution. Two hours after drinking the glucose solution, a second blood glucose measurement is performed. A second blood glucose measurement of below 140 mg/dL is considered normal, a second blood glucose measurement between 140 mg/L and 200 mg/dL is considered to represent impaired glucose tolerance condition, and a second blood glucose measurement that is greater than 200 mg/dL is considered to indicate diabetic condition. In addition to the lengthy process for performing the oral glucose tolerance test, many find the need to drink the glucose solution as part of the test to be distasteful and undesirable. In some contexts, these drawbacks are significant enough to cause an individual to forego performing the oral glucose tolerance test, potentially resulting in undiagnosed pre-diabetic or diabetic conditions. Accordingly, there is an ongoing desire and an important need to improve glycemic control of individuals and in particular, to accurately and timely diagnose the onset of diabetes as well as diabetic condition itself in a way that is convenient, accurate, and timely.

### SUMMARY

Embodiments of the present disclosure include methods, devices and systems for determining fasting glucose level information, that include performing meal start time determination for each one day time period, determining a plurality of fasting metrics, each fasting metric corresponding to a respective one day time period, determining an overall fasting metric, and generating fasting glucose level information.

Certain embodiments for the meal start time determination include retrieving from a storage unit a meal start time of day period, retrieving an insulin delivery data within the meal start time of day period, determining a potential meal start time within the meal start time of day period, comparing the insulin delivery information with the potential meal start time, and setting the meal start time as the determined potential meal start time when the insulin delivery information correlates with the potential meal start time based on the comparison.

Certain embodiments for determining fasting glucose level information includes an apparatus with one or more processors, and a storage unit operatively coupled to the one or more processors and configured to store instructions which, when executed by the one or more processors, controls the one or more processors to perform meal start time determination for each one day time period, to determine a plurality of fasting metrics, each fasting metric corresponding to a respective one day time period, to determine an overall fasting metric, and to generate fasting glucose level information.

A system for determining fasting glucose level information, in certain embodiments, includes an in vivo glucose sensor operatively coupled to the one or more processors, the glucose sensor having a portion positioned under a skin surface and in contact with bodily fluid and configured to generate signals corresponding to monitored glucose level in the bodily fluid, a glucose monitor operatively coupled to the glucose sensor to process the signals from the glucose sensor and to generate glucose measurement data, a data processing device in signal communication with the glucose monitor to receive the glucose measurement data, the data processing device including: one or more processors, and a storage unit operatively coupled to the one or more processors and configured to store instructions which, when executed by the one or more processors, controls the one or more processors to perform meal start time determination for each one day time period, to determine a plurality of fasting metrics, each fasting metric corresponding to a respective one day time period, to determine an overall fasting metric, and to generate fasting glucose level information.

The system for determining fasting glucose level information in certain embodiments includes an insulin delivery device in signal communication with the data processing device, where the insulin delivery device is configured to provide insulin delivery information to the data processing device, and further, where the data processing device is configured to retrieve from the storage unit a meal start time of day period, to identify an insulin delivery information within the meal start time of day period received from the insulin delivery device, to determine a potential meal start time within the meal start time of day period, to compare the insulin delivery information with the potential meal start time, and to set the meal start time as the determined potential meal start time when the insulin delivery information correlates with the potential meal start time based on the comparison. For example, insulin delivery devices (e.g., devices integrating an infusion device therein) to administer insulin therapy to patients may administer and modify basal profiles, as well as determine appropriate boluses for administration based on, among others, the detected analyte levels.

Embodiments of the present disclosure further include methods, devices and systems for performing post-prandial glucose level analysis that include performing meal start time determination for each one day time period, determining a post-prandial metric for each day based on the meal start time for the corresponding day, determining an overall post-prandial metric from the plurality of the post-prandial metric for each day, and generating a post-prandial glucose level information.

Certain embodiments for determining the post-prandial metric for each day includes retrieving glucose data for a first time period relative to the meal start time, determining a pre-meal glucose parameter from the retrieved glucose data for the first time period, retrieving glucose data for a second time period relative to the meal start time, determining a post-meal glucose parameter from the retrieved glucose data for the second time period, and determining a post-prandial glucose metric from the pre-meal and post-meal glucose parameters.

An apparatus for performing post-prandial glucose level information analysis, in certain embodiments, includes one or more processors, and a storage unit operatively coupled to the one or more processors and configured to store instructions which, when executed by the one or more processors, controls the one or more processors to perform meal start time determination for each one day time period, to determine a post-prandial metric for each day based on the meal start time for the corresponding day, to determine an overall post-prandial metric from the plurality of the post-prandial metric for each day, and to generate a post-prandial glucose level information.

A system for performing post-prandial glucose level information analysis, in certain embodiments, includes an in vivo glucose sensor operatively coupled to the one or more processors, the glucose sensor having a portion positioned under a skin surface and in contact with bodily fluid and configured to generate signals corresponding to monitored glucose level in the bodily fluid, a glucose monitor operatively coupled to the glucose sensor to process the signals from the glucose sensor and to generate glucose measurement data, a data processing device in signal communication with the glucose monitor to receive the glucose measurement data, the data processing device including: one or more processors, and a storage unit operatively coupled to the one or more processors and configured to store instructions which, when executed by the one or more processors, controls the one or more processors to perform meal start time determination for each one day time period, to determine a post-prandial metric for each day based on the meal start time for the corresponding day, to determine an overall post-prandial metric from the plurality of the post-prandial metric for each day, and to generate a post-prandial glucose level information.

In this manner, accurate and reliable fasting glucose determination and post-prandial glucose tolerance determination are provided to the individuals for pre-diabetes or diabetic condition diagnosis without the need for the individuals to perform inconvenient and cumbersome fasting glucose test, nor to require consumption of distasteful glucose solution as part of an oral glucose tolerance test.

These and other features, objects and advantages of the present disclosure will become apparent to those persons skilled in the art upon reading the details of the present disclosure as more fully described below.

### BRIEF DESCRIPTION OF THE DRAWINGS

FIG. 1A is a system for providing fasting glucose analysis and post-prandial glucose tolerance analysis in accordance with one embodiment of the present disclosure;

FIG. 1B is a data flow for providing fasting glucose analysis and post-prandial glucose tolerance analysis of the overall system of FIG. 1A in accordance with one embodiment of the present disclosure;

FIG. 1C is an overall system for providing fasting glucose analysis and post-prandial glucose tolerance analysis in accordance with another embodiment of the present disclosure;

FIG. 2 is an exemplary output display of the fasting glucose analysis and post-prandial glucose tolerance analysis from the overall systems of FIGS. 1A and 1C in accordance with one embodiment of the present disclosure;

FIG. 3 is an exemplary time line for the fasting glucose analysis and the post-prandial glucose analysis in accordance with one embodiment of the present disclosure;

FIG. 4A is a flowchart illustrating a routine to determine fasting glucose level information in accordance with one embodiment of the present disclosure;

FIG. 4B is a flowchart illustrating a routine to perform daily meal start determination in the fasting glucose level information determination routine of FIG. 4B in accordance with one embodiment of the present disclosure;

FIG. 5A is a flowchart illustrating a routine to determine post-prandial glucose level information in accordance with one embodiment of the present disclosure; and

FIG. 5B is a flowchart illustrating a routine to determine a post-prandial metric for each day based on the determined meal start times in the post-prandial glucose level information determination routine of FIG. 5A in accordance with one embodiment of the present disclosure.

### DETAILED DESCRIPTION

Before the present disclosure is described in detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges as also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The figures shown herein are not necessarily drawn to scale, with some components and features being exaggerated for clarity.

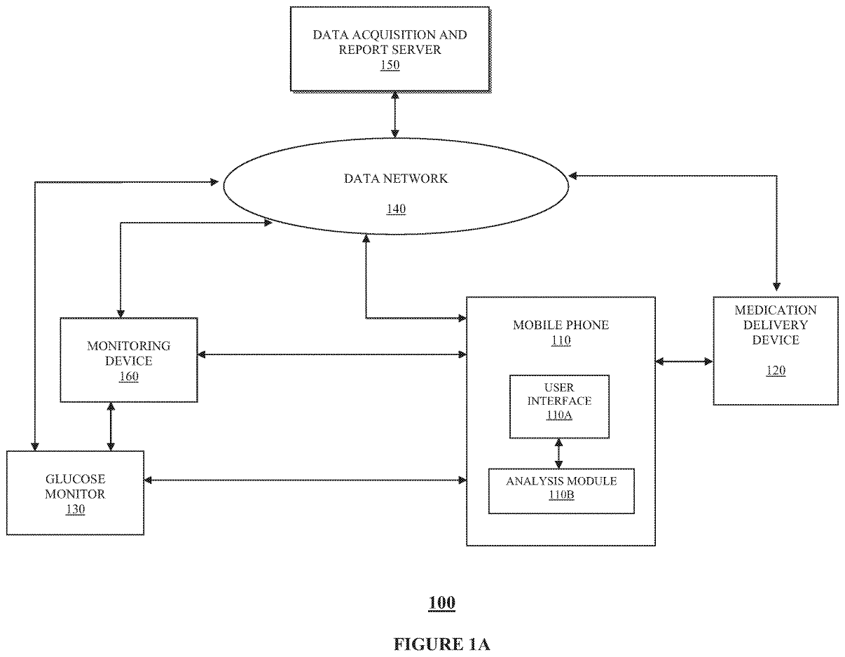

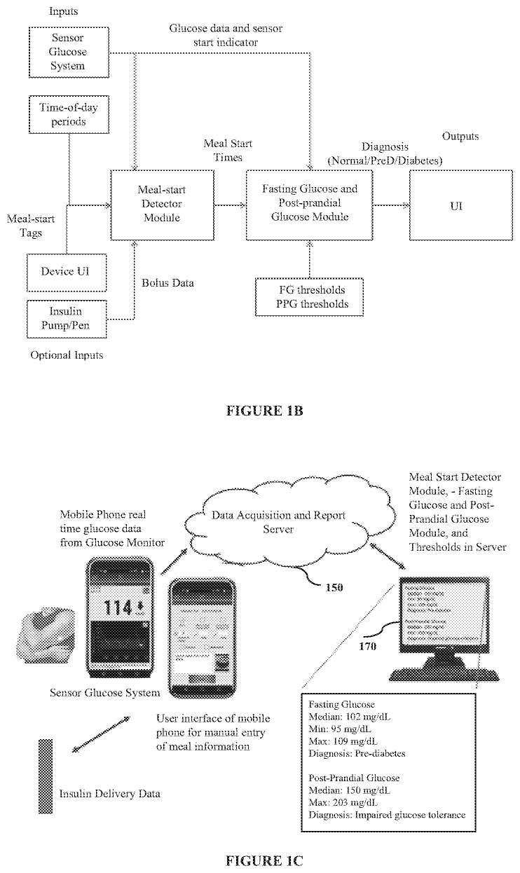

FIG. 1A is a system for providing fasting glucose analysis and post-prandial glucose tolerance analysis in accordance with one embodiment of the present disclosure. Referring to the FIG. 1A, diagnosis and therapy system 100, in certain embodiments, includes a data network 140 operatively coupled to data acquisition and report server 150. As shown, diagnosis and therapy system 100 also includes mobile phone 110 including user interface 110A and analysis module 110B programmed in the mobile phone 110 as a software application (“App”) that is executable by any processor controlled device, and in particular, a smart phone with communication capabilities to receive, analyze, transfer, transmit, display or output or nonactionable, actionable information, for example, including medical condition diagnosis based on the received glucose data analysis. In certain embodiments, the App is installed in the mobile phone 110 as a downloaded executable file over data network 140 from server 150. As discussed in further detail below, in certain embodiments, the App is configured to provide or output on the user interface 110A diagnosis information such as pre-diabetes condition, and/or impaired glucose tolerance condition from the real time glucose level information.

Referring to FIG. 1A, also included in diagnosis and therapy system 100 is glucose monitor 130 and monitoring device 160 each operatively coupled to and in bi-directional communication with data network 140 and mobile phone 110. Also shown in FIG. 1A is medication delivery device 120 also operatively coupled to and in bi-directional communication with data network 140 and mobile phone 110. The communication between mobile phone 110, medication delivery device 120, glucose monitor 130, monitoring device 160 and data network 140 includes one or more wireless communication, wired communication, Bluetooth® communication, WiFi data communication, radio frequency identification (RFID) enabled communication, Zigbee® communication, or any other suitable data communication that optionally supports data encryption/decryption, data compression, data decompression and the like.

Glucose monitor 130 shown in FIG. 1A includes one or more in vivo glucose sensors, each with a portion configured to be in fluid contact with bodily fluid of a user under a skin surface, and coupled to sensor electronics attached or mounted on the skin surface for processing signals from the glucose sensor and communicating the processed glucose signals to one or more of mobile phone 110, monitoring device 160, data acquisition and report server 150, and medication delivery device 120 over a direct communication link with the one or more of these devices, or alternatively, over the data network 140. Further, monitoring device 160 includes, in certain embodiments, compact, handheld data processing devices that are configured for communication with the glucose monitor 130 and for further processing data received from the glucose monitor 130, and optionally to program, diagnose, or otherwise monitor the operation of glucose monitor 130. Additional details on the glucose monitor 130 including glucose sensor and sensor electronics and monitoring device 160 can be found in U.S. Pat. No. 6,175,752 and US Patent Publication No. 2011/0213225, both assigned to the assignee of the present application, Abbott Diabetes Care Inc., Alameda, California, the disclosures of each of which are incorporated herein by reference for all purposes.

FIG. 1B is a data flow for providing fasting glucose analysis and post-prandial glucose tolerance analysis of the overall system of FIG. 1A in accordance with one embodiment of the present disclosure. Referring to FIG. 1B, in certain embodiments, one or more of monitored glucose level information, time of day period information, meal start tag/time stamp information, insulin delivery time information and/or other user initiated information is provided to the analysis module 110B and/or to the data acquisition and report server 150 to perform fasting glucose analysis and/or post-prandial glucose tolerance analysis. As shown in FIG. 1B, in certain embodiments, the analysis module 110B and/or the data acquisition and report server 150 includes a meal start detector module, a fasting glucose and post-prandial glucose module as well as one or more storage units such as non-volatile memory devices to store received data and one or more threshold parameters associated with the fasting glucose analysis and/or the-post prandial glucose analysis. Referring again to FIG. 1B, the result(s) of the fasting glucose analysis and/or post-prandial glucose tolerance analysis is presented on the user interface 110A of the mobile telephone 110, a user interface of the monitoring device 160 (FIG. 1) and/or a computer terminal 170 (FIG. 1C) as discussed in conjunction with FIG. 1C below.

FIG. 1C is a system for providing fasting glucose analysis and post-prandial glucose tolerance analysis in accordance with embodiments of the present disclosure. In certain embodiments, some or all of the functions of the App related to the fasting glucose analysis and/or the post-prandial glucose analysis are implemented by data acquisition and report server 150 to provide diagnosis information such as pre-diabetes condition, and/or impaired glucose tolerance condition from the real time glucose level information. As shown in FIG. 1C, information from glucose monitor 130 and/or monitoring device 160 and/or medication delivery device 120 are pushed automatically and/or periodically to data acquisition and report server 150 for analysis. In certain embodiments, information/data from one or more of glucose monitor 130, monitoring device 160 and medication delivery device 120 is communicated to mobile phone 110. Upon receipt of the information/data, mobile phone 110 is configured to communicate the received information/data to data acquisition and report server 150 for analysis. In this manner, in an embodiment, mobile phone 110 is configured to operate as a data conduit or transfer device to collect information/data and transfer the received information/data to data acquisition and report server 150 for analysis.

Further, in certain embodiments, one or more of the glucose monitor 130, monitoring device 160 and medication delivery device 120 are each configured with the App for execution therein locally. In other words, embodiments of the present disclosure include one or more of the glucose monitor 130, monitoring device 160, medication delivery device 120 that includes analysis module 110B for processing glucose data, medication delivery information, time of day period data, among others, and to generate diagnosis information such as pre-diabetes condition and/or impaired glucose tolerance condition based on the real time glucose level information.

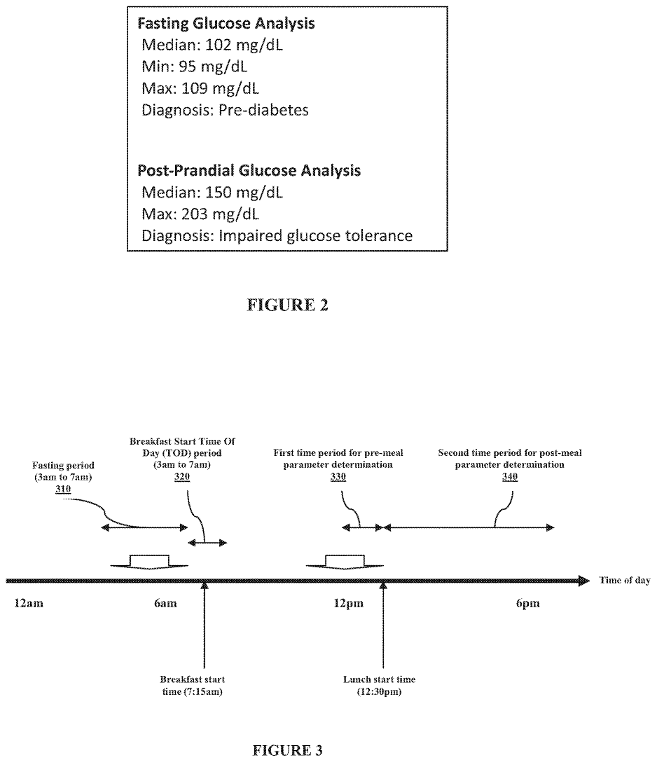

FIG. 2 is an example output display of the fasting glucose analysis and post-prandial glucose tolerance analysis in accordance with one embodiment of the present disclosure. Referring to FIG. 2, in certain embodiments, the App in analysis module 110B of the mobile phone 110 performs fasting glucose level analysis and post-prandial glucose level analysis (to determine glucose tolerance level), and outputs the diagnosis information on user interface 110A. In another embodiment, the data acquisition and report server 150 performs fasting glucose level analysis and post-prandial glucose level analysis (to determine glucose tolerance level), and pushes the results of the analysis to the display of the physician's computer terminal 170 (FIG. 1C) to provide diagnosis information. As shown, the results of the fasting glucose level analysis includes, for example, but not limited to, median glucose level (102 mg/L), minimum glucose level (95 mg/dL) and maximum glucose level (109 mg/dL), with a diagnosis indication showing “pre-diabetes” condition. Also shown in FIG. 2 is the post-prandial glucose level analysis including, for example, median glucose level (150 mg/dL), and maximum glucose level (203 mg/dL) with a corresponding diagnosis indication of “impaired glucose tolerance” condition.

Referring again to FIG. 2, while mobile phone 110 is provided with user interface 110A, each of the medication delivery device 120, monitoring device 160, and glucose monitor 130, in certain embodiments, includes a display or user interface that is configured to output the same information shown in FIG. 2 locally on the respective devices.

In embodiments, medication delivery device 120 may include an infusion device such as an insulin infusion pump or the like, which may be configured to administer insulin to patients. In alternatives, a device may be configured to integrate an infusion device therein so that the device is configured to administer insulin (or other appropriate drug) therapy to patients, for example, for administering and modifying basal profiles, as well as for determining appropriate boluses for administration based on the detected analyte levels received from a source, such as the sensors of the glucose monitor.

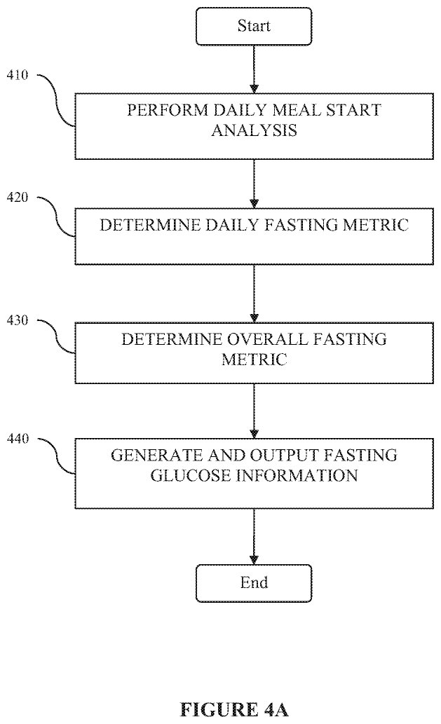

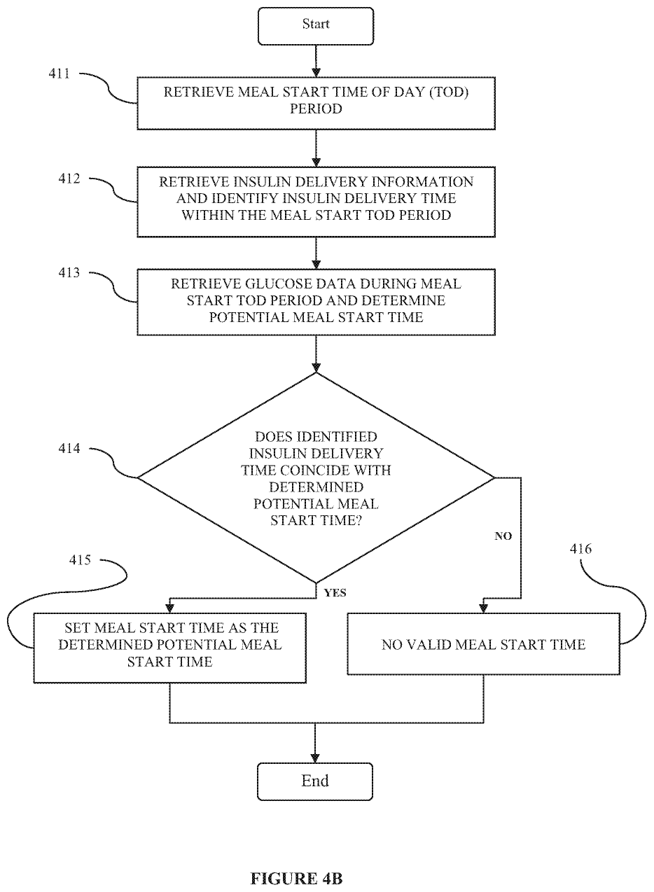

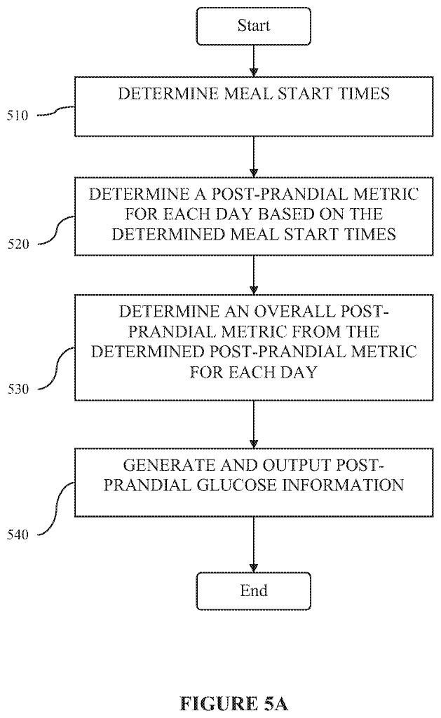

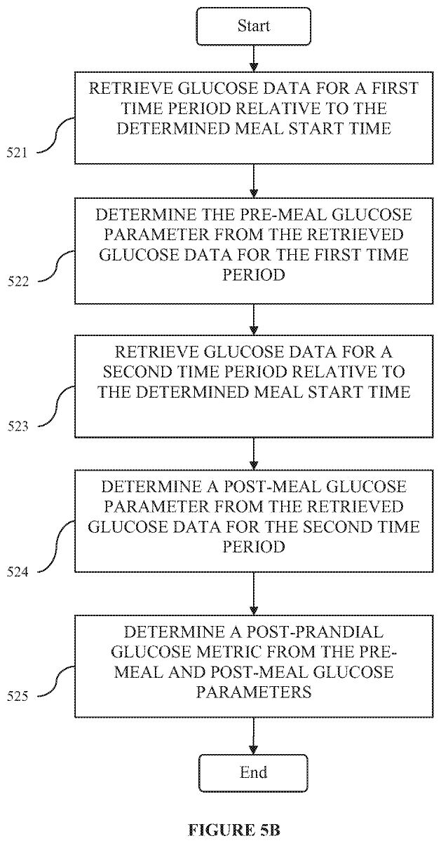

FIG. 3 is an exemplary time line for the fasting glucose analysis and the post-prandial glucose analysis in accordance with one embodiment of the present disclosure. As discussed in further detail in conjunction with FIGS. 4A-4B and 5A-5B, each of the fasting glucose level analysis and the post-prandial glucose level analysis uses glucose level information, for example from the in vivo glucose sensor of glucose monitor 130 (FIG. 1) positioned in fluid contact with bodily fluid under the skin surface, over a sensor wear (sensor life) time period-for the duration of the glucose sensor wear of 7 days, 10 days, 14 days, one month, two months, three months, six months or more. In certain embodiments, the glucose data from the glucose sensor are received or collected at a predetermined time interval such as every minute, every five minutes, every 10 minutes, every 15 minutes, or more, and stored in a memory or storage device of the glucose monitor 130 and thereafter communicated to the one or more monitoring device 160, mobile phone 110, medication delivery device 120 and the data acquisition and report server 150.

In embodiments, the bodily fluid may be dermal fluid or interstitial fluid.

Referring again to FIG. 3, and as discussed in further detail below, in certain embodiments, meal start time of day (TOD) period associated with a meal start time (e.g., breakfast start time of day (TOD) period 320 shown in FIG. 3) is stored in one or more storage devices of one or more of monitoring device 160, mobile phone 110, medication delivery device 120 and the data acquisition and report server 150. Also shown in FIG. 3 is fasting period 310 associated with breakfast start time. In certain embodiments, and as discussed in further detail below, the breakfast start TOD period 320, fasting period 310, and breakfast start time (7:15 am) are used with glucose level information obtained from an in vivo glucose sensor to perform fasting glucose analysis.

Referring still to FIG. 3 and as discussed in further detail below, in certain embodiments, meal start time is determined for each meal during a 24 hour period. Then, two time periods as shown in FIG. 3 (for example, in conjunction with lunch start time) such as a first time period for pre-meal parameter determination 330 and a second time period for post-meal parameter determination 340 are retrieved from the storage unit or memory device of mobile phone 110 or at data acquisition and report server 150, for example, and used in conjunction with the determined meal start time to perform post-prandial glucose analysis. Additional detailed description of the automatic or programmed meal start detection routine is described in PCT Patent Publication No. WO 2015/153482, assigned to the assignee of the present disclosure, and the disclosure of which is incorporated by reference in its entirety for all purposes.

FIG. 4A is a flowchart illustrating a routine to determine fasting glucose level information in accordance with one embodiment of the present disclosure. Referring to FIG. 4A, fasting glucose level analysis in certain embodiments include performing daily meal start determination analysis (410) to determine the start time of the first meal of the day. Thereafter a fasting metric for each day with the start time of the first meal of the day is determined (420).

In certain embodiments, the daily fasting metric is determined, for example, by retrieving glucose data received from the glucose monitor 130 (FIG. 1) for a fasting period (FIG. 3) corresponding to the determined meal start time. In certain embodiments, the fasting period spans the time period from 4 hours and 15 minutes before the meal start time to 15 minutes before the meal start time. That is, in one embodiment, the fasting period is a 4 hour period before the meal start time that ends 15 minutes before the meal start time. While 4 hours and 15 minutes are used herein, these values for the fasting period are non-limiting examples only and are not intended to limit the scope of the present disclosure with these values. Rather, within the scope of the present disclosure, the fasting time period can including other time periods (for example, 3 hours, 3.5 hours, 4 hours, 4.5 hours, 5 hours, 5.5 hours, 6 hours, and/or any other suitable time ranges including intervening time ranges) that precede the determined meal start time. Further, the 15 minutes preceding the meal start time by which the fasting period ends is described herein solely as a non-limiting example, and other suitable time ranges can be used within the scope of the present disclosure (for example, 10 minutes, 15 minutes, 20 minutes, 25 minutes, or any other suitable time ranges including intervening times).

Referring back to FIG. 4A, after retrieving the glucose data for the fasting period (FIG. 3) corresponding to the determined meal start time, the daily fasting metric is determined, in certain embodiments, by calculating the median of the retrieved glucose data for the fasting period. In other embodiments, the daily fasting metric is determined by identifying the glucose level at a predetermined time preceding the meal start time (for example, at 15 minutes before the determined meal start time). Alternatively, daily fasting metric is determined by taking the mean, minimum, maximum, or other suitable metric of the glucose data over the fasting period.

With the determined daily fasting metric, an overall fasting metric from the plurality of daily fasting metric is determined for the time period spanning the number of days for which daily fasting metric is determined (430). Using the overall fasting metric, fasting glucose level information as shown in FIG. 2 is generated and output to the user interface (440). In certain embodiments, the determination of the overall fasting metric across all days that have a valid daily fasting metric includes determining one or more of a median, mean, standard deviation, inter-quartile range (IQR), a minimum, and a maximum of the fasting metric for all days with valid daily fasting metric.

FIG. 4B is a flowchart illustrating a routine to perform daily meal start determination in the fasting glucose level information determination routine of FIG. 4B in accordance with one embodiment of the present disclosure. Referring to FIG. 4B, in certain embodiments, meal start determination includes retrieving meal start TOD period from memory or from a previously stored setting (411). Thereafter, insulin delivery information is retrieved and insulin delivery time within the meal start TOD period is identified (412). Also, glucose data during the meal start TOD period is retrieved and a potential meal start time is determined by analyzing the retrieved glucose data during the meal start TOD period (413). In certain embodiments, the potential meal start time is determined from the retrieved glucose data by isolating or identifying the beginning of a sustained glucose level change in direction. In certain embodiments, potential meal start times are determined by identifying upward trend in glucose following a rapid acting insulin delivery marker (e.g., bolus delivery time), identifying an upward trend in the monitored glucose level following a meal marker, or identifying the insulin delivery marker or the meal marker as the start of the meal.

Referring back to FIG. 4B, the retrieved insulin delivery time is compared with the potential meal start time to determine whether the retrieved insulin delivery time correlates with the potential meal start time (414). If the retrieved insulin delivery time does not correlate with the potential meal start time, then no valid meal start time is identified (416) that corresponds with the meal start TOD period. On the other hand, if the retrieved insulin delivery time correlates or coincides with the potential meal start time, then the meal start time is set to the determined potential meal start time (415).

In certain embodiments, when there is no insulin delivery information available, meal start tag (user initiated) can be used in the analysis by, for example, comparing the time information associated with the meal start tag to determine whether the potential meal start time correlates with the meal start tag. If the potential meal start time correlates with the meal start tag, the potential meal start time is identified as the meal start time for the retrieved meal start TOD period. In certain embodiments, the meal start time is determined based on a user initiated meal event tag using the user interface 110A of mobile phone 110, for example, when the user manually indicates the start of the meal event. In still other embodiments, the time information associated with the meal start tag can be used as the meal start time. Within the scope of the present disclosure, insulin delivery time or time information for the meal start tag can be used, depending on which is available for the meal start TOD period, to confirm the meal start time.

In this manner, referring back to FIGS. 4A-4B, fasting glucose level information is determined by analyzing the stored glucose measurement data and provided to the user or the physician. With the fasting glucose level information, the App may provide diagnosis information, such as, for example, “pre-diabetes” condition (FIG. 2). With the diagnosis information, the user is able to make informed and timely corrective action, under the guidance of a physician, for example, to take medication, modify diet, implement exercise regimen, and the like.

In certain embodiment, the App may be configured to discard the identified and stored glucose measurement from the analysis data set that corresponds to the first 24 hour time period of the glucose sensor wear. This feature is configured to improve the accuracy of the fasting glucose level analysis by excluding glucose data that may suffer from aberrant signal such as can be observed upon sensor initiation periods, for example signal attenuation error. Additional detailed information on signal attenuation is provided in U.S. Pat. No. 8,583,205, assigned to the assignee of the present disclosure, the disclosure of which is incorporated by reference in its entirety for all purposes.

FIG. 5A is a flowchart illustrating a routine to determine post-prandial glucose level information in accordance with one embodiment of the present disclosure. Referring to FIG. 5A, for each day in the post-prandial glucose level information analysis time period, a corresponding meal start time is determined (510). In certain embodiments, the meal start time is determined as described above in conjunction with FIGS. 4A and 4B and thus, not repeated here. In this manner, for each day in the analysis time period, the meal start time for breakfast, lunch and dinner are determined. Thereafter, a post-prandial metric for each day is determined based on the meal start times (520). As discussed in further detail in conjunction with FIG. 5B below, the post-prandial metric for each day is determined using select glucose level information associated with the meal start times.

Referring to FIG. 5A, after determining the post-prandial metric for each day of the analysis time period, an overall post-prandial metric is determined using the post-prandial metric for each day (530), and the post-prandial glucose information from the overall post-prandial metric is generated and/or output to the user or the physician (540). For example, referring to FIG. 2, post-prandial glucose analysis includes a visual output of the median glucose level (e.g., 150 mg/dL) and maximum glucose level (e.g., 203 mg/dL), and a resulting diagnosis of “impaired glucose tolerance”.

FIG. 5B is a flowchart illustrating a routine to determine a post-prandial metric for each day based on the meal start times in the post-prandial glucose level information determination routine of FIG. 5A in accordance with one embodiment of the present disclosure. Referring to FIG. 5B, for each determined meal start time, glucose data for a first time period relative to the meal start time is retrieved (521). For example, glucose data for 30 minutes (or other suitable time periods) before the meal start time is retrieved. While 30 minutes is used herein, within the scope of the present disclosure, other time periods can be used including, but not limited to, 20 minutes, 25 minutes, 30 minutes, 35 minutes, 40 minutes, 45 minutes, one hour or greater, or any other intervening time periods.

Thereafter, a pre-meal glucose parameter is determined from the glucose data for the first time period (522). In one embodiment, the pre-meal glucose parameter includes the median of the glucose data for the first time period. In other embodiments, the pre-meal glucose parameter includes a single glucose level identified within the first time period, or a median of the glucose data in the first time period. Referring back to FIG. 5B, glucose data for a second time period relative to the meal start time is retrieved (523). For example, glucose data for a 6 hour time period from the meal start time is retrieved for analysis. While 6 hour time period is selected here, within the scope of the present disclosure, other time periods can be used including, but not limited to, 4 hour, 5 hour, 6 hour, 7 hour or more, or any intervening time period. In another embodiment, the second time period starts with the meal start time and ends immediately prior to the next, subsequent meal start time. For example, the glucose data in the second time period includes all glucose data collected from the meal start time through the last glucose data point available before the next meal start time.

Referring again to FIG. 5B, a post-meal glucose parameter from the retrieved glucose data for the second time period is determined (524). In one embodiment, the post-meal glucose parameter includes a post-meal peak parameter determined by calculating the maximum glucose level from the glucose data in the second time period. In other embodiments, the post-meal glucose parameter includes a mean or median of the glucose data from the second time period. The post-prandial glucose metric is determined from the pre-meal glucose parameter and the post-meal glucose parameters (525). For example, in one embodiment, the post-prandial glucose metric includes a peak-difference metric, which is the difference between the post meal peak parameter (the maximum glucose level from the glucose data in the second time period) and the pre-meal glucose parameter (the median of the glucose data for the first time period).

In this manner, post-prandial glucose level analysis is performed in accordance with various embodiments of the present disclosure using glucose data received from glucose monitor 130 (FIG. 1). Referring again to FIG. 2, the diagnosis information output from the post prandial glucose level analysis is presented, e.g., visually, audibly, and the like, where the median glucose level (150 mg/dL) and maximum glucose level (203 mg/dL) are output with a diagnosis of “impaired glucose tolerance”, or other notification. Similar to the diagnosis based on the fasting glucose level analysis described above, the diagnosis from the post prandial glucose level analysis described here provides physiological information from which the user can take corrective action, optionally under the guidance of a physician, to improve glycemic control by, for example, modifying diet, implementing an exercise regimen, and/or with medication. Output information, such as the diagnosis information, may be used for therapy-, administration-or treatment-related decisions.

Since the analysis relies on glucose measurement data from the glucose monitor 130 (FIG. 1) to provide diagnosis for identifying pre-diabetic condition or diabetic condition, the inconvenience of actual fasting as required for the conventional fasting glucose test, or the distasteful and undesirable experience of drinking glucose solution, and performing multiple blood glucose measurements as required for the conventional glucose tolerance test, are obviated by the embodiments of the present disclosure.

An apparatus for determining fasting glucose level information may comprise one or more processors; and a storage unit operatively coupled to the one or more processors and configured to store instructions which, when executed by the one or more processors, controls the one or more processors to perform meal start time determination for each one day time period, to determine a plurality of fasting metrics, each fasting metric corresponding to a respective one day time period, to determine an overall fasting metric, and to generate fasting glucose level information.

In an embodiment, the one or more processors executing the stored instructions to perform the meal start time determination is configured to retrieve from the storage unit a meal start time of day period, to retrieve an insulin delivery information within the meal start time of day period, to determine a potential meal start time within the meal start time of day period, to compare the insulin delivery information with the potential meal start time, and to set the meal start time as the determined potential meal start time when the insulin delivery information correlates with the potential meal start time based on the comparison. The retrieved insulin delivery information may include insulin delivery start time. The meal start time may be set as the potential meal start time when the insulin delivery information coincides with the potential meal start time. The retrieved insulin delivery information may include insulin delivery start time.

In another embodiment, the one or more processors executing the stored instructions to determine the potential meal start time is configured to retrieve glucose data collected during the meal start time of day period, and to determine the potential meal start time based on the retrieved glucose data.

In embodiments, the one or more processors executing the stored instructions to determine each fasting metric is configured to retrieve a plurality of glucose data collected over a fasting period, the fasting period preceding the meal start time, and to determine a median of the retrieved plurality of glucose data over the fasting period. The fasting period may span a predetermined time period that begins and ends before the meal start time. The end of the predetermined time period for the fasting period may precede the beginning of the meal start time by a predetermined amount of time. The one or more processors may be configured to apply a function to the determined fasting metric to determine the overall fasting metric. The function may include one or more of a median, a mean, a standard deviation, an interquartile range, a minimum, or a maximum.

In an aspect, the apparatus may further comprise a user interface is operatively coupled to the one or more processors, wherein the one or more processors is configured to generate and output diagnosis information based on the fasting glucose level information. The generated diagnosis information may provide an indication of diabetic condition. The diagnosis information may include a pre-diabetes condition.

In another aspect, the one or more processors may be configured to detect a meal start tag based on a user input to perform the meal start time determination for each one day time period.

In aspects, the apparatus may further include an in vivo glucose sensor operatively coupled to the one or more processors, the glucose sensor having a portion positioned under a skin surface and in contact with bodily fluid and configured to generate signals corresponding to monitored glucose level in the bodily fluid and that are stored in the storage unit as glucose measurement data. The bodily fluid may include dermal fluid, or interstitial fluid. The glucose sensor may include a plurality of electrodes including a working electrode comprising an analyte-responsive enzyme bonded to a polymer disposed on the working electrode. The analyte-responsive enzyme may be chemically bonded to the polymer disposed on the working electrode. The working electrode may comprise a mediator bonded to the polymer disposed on the working electrode. The mediator may be crosslinked with the polymer disposed on the working electrode.

In another aspect, the glucose sensor may include a plurality of electrodes including a working electrode comprising a mediator bonded to a polymer disposed on the working electrode.

A system for determining fasting glucose level information comprises an in vivo glucose sensor having a portion positioned under a skin surface and in contact with bodily fluid and configured to generate signals corresponding to monitored glucose level in the bodily fluid; a glucose monitor operatively coupled to the glucose sensor to process the signals from the glucose sensor and to generate glucose measurement data; and a data processing device in signal communication with the glucose monitor to receive the glucose measurement data, the data processing device including: one or more processors; and a storage unit operatively coupled to the one or more processors and configured to store instructions which, when executed by the one or more processors, controls the one or more processors to perform meal start time determination for each one day time period, to determine a plurality of fasting metrics, each fasting metric corresponding to a respective one day time period, to determine an overall fasting metric, and to generate fasting glucose level information.

In an embodiment, the data processing device includes a data server located remotely from the glucose monitor. The data processing device may include a mobile telephone configured to receive the generated glucose measurement data from the glucose monitor.

In embodiments, the system may include an insulin delivery device in signal communication with the data processing device, the insulin delivery device configured to provide insulin delivery information to the data processing device, and further, wherein the data processing device is configured to retrieve from the storage unit a meal start time of day period, to identify an insulin delivery information within the meal start time of day period received from the insulin delivery device, to determine a potential meal start time within the meal start time of day period, to compare the insulin delivery information with the potential meal start time, and to set the meal start time as the determined potential meal start time when the insulin delivery information correlates with the potential meal start time based on the comparison.

A method of performing post-prandial glucose level information analysis includes performing meal start time determination for each one day time period; determining a post-prandial metric for each day based on the meal start time for the corresponding day; determining an overall post-prandial metric from the plurality of the post-prandial metric for each day; and generating a post-prandial glucose level information.

In embodiments, determining the post-prandial metric for each day includes retrieving glucose data for a first time period relative to the meal start time; determining a pre-meal glucose parameter from the retrieved glucose data for the first time period; retrieving glucose data for a second time period relative to the meal start time; determining a post-meal glucose parameter from the retrieved glucose data for the second time period; and determining a post-prandial glucose metric from the pre-meal and post-meal glucose parameters. The first time period may end before the beginning of the meal start time, and the second time period starts after the end of the meal start time. The pre-meal glucose parameter may be determined by applying a function to the retrieved glucose data for the first time period. The function may include one or more of a median, a mean, a standard deviation, an interquartile range, a minimum, or a maximum.

The post-meal glucose parameter may be determined by applying a function to the retrieved glucose data for the second time period. The function may include one or more of a median, a mean, a standard deviation, an interquartile range, a minimum, or a maximum.

In an embodiment, performing the meal start time determination includes: retrieving from a storage unit a meal start time of day period; retrieving an insulin delivery information within the meal start time of day period; determine a potential meal start time within the meal start time of day period; comparing the insulin delivery information with the potential meal start time; and setting the meal start time as the determined potential meal start time when the insulin delivery information correlates with the potential meal start time based on the comparison.

In an aspect, an apparatus for forming post-prandial glucose level information analysis comprises one or more processors; and a storage unit operatively coupled to the one or more processors and configured to store instructions which, when executed by the one or more processors, controls the one or more processors to perform meal start time determination for each one day time period, to determine a post-prandial metric for each day based on the meal start time for the corresponding day, to determine an overall post-prandial metric from the plurality of the post-prandial metric for each day, and to generate a post-prandial glucose level information.

In an embodiment, the or more processors executing the stored instructions to determine the post-prandial metric for each day may be configured to retrieve glucose data for a first time period relative to the meal start time, to determine a pre-meal glucose parameter from the retrieved glucose data for the first time period, to retrieve glucose data for a second time period relative to the meal start time, to determine a post-meal glucose parameter from the retrieved glucose data for the second time period, and to determine a post-prandial glucose metric from the pre-meal and post-meal glucose parameters. The first time period ends before the beginning of meal start time, and the second time period starts after the end of the meal start time. The pre-meal glucose parameter may be determined by applying a function to the retrieved glucose data for the first time period. The function may include one or more of a median, a mean, a standard deviation, an interquartile range, a minimum, or a maximum.

In an aspect, the post-meal glucose parameter may be determined by applying a function to the retrieved glucose data for the second time period. The function may include one or more of a median, a mean, a standard deviation, an interquartile range, a minimum, or a maximum.

A system for performing post-prandial glucose level information analysis, comprises: an in vivo glucose sensor having a portion positioned under a skin surface and in contact with bodily fluid and configured to generate signals corresponding to monitored glucose level in the bodily fluid; a glucose monitor operatively coupled to the glucose sensor to process the signals from the glucose sensor and to generate glucose measurement data; and a data processing device in signal communication with the glucose monitor to receive the glucose measurement data, the data processing device including: one or more processors; and a storage unit operatively coupled to the one or more processors and configured to store instructions which, when executed by the one or more processors, controls the one or more processors to perform meal start time determination for each one day time period, to determine a post-prandial metric for each day based on the meal start time for the corresponding day, to determine an overall post-prandial metric from the plurality of the post-prandial metric for each day, and to generate a post-prandial glucose level information.

In aspects, the data processing device determining the post-prandial metric for each day may be configured to retrieve glucose data for a first time period relative to the meal start time, to determine a pre-meal glucose parameter from the retrieved glucose data for the first time period, to retrieve glucose data for a second time period relative to the meal start time, to determine a post-meal glucose parameter from the retrieved glucose data for the second time period, and to determine a post-prandial glucose metric from the pre-meal and post-meal glucose parameters.

In an embodiment, the data processing device may include a data server located remotely from the glucose monitor.

In another embodiment, the data processing device may include a mobile telephone configured to receive the generated glucose measurement data from the glucose monitor.

In an aspect, the system includes an insulin delivery device in signal communication with the data processing device, the insulin delivery device configured to provide insulin delivery information to the data processing device, and further, wherein the data processing device performing the meal start time determination is configured to retrieve from the storage unit a meal start time of day period, to receive from the insulin delivery device the insulin delivery information within the meal start time of day period, to determine a potential meal start time within the meal start time of day period, to compare the insulin delivery information with the potential meal start time, and to set the meal start time as the determined potential meal start time when the insulin delivery information correlates with the potential meal start time based on the comparison.

In embodiments, the one or more processors executing the stored instructions to perform the meal start time determination may be configured to retrieve from the storage unit a meal start time of day period, to retrieve an insulin delivery information within the meal start time of day period, to determine a potential meal start time within the meal start time of day period, to compare the insulin delivery information with the potential meal start time, and to set the meal start time as the determined potential meal start time when the insulin delivery information correlates with the potential meal start time based on the comparison.

Various other modifications and alterations in the structure and method of operation of this disclosure will be apparent to those skilled in the art without departing from the scope and spirit of the embodiments of the present disclosure. Although the present disclosure has been described in connection with particular embodiments, it should be understood that the present disclosure as claimed should not be unduly limited to such particular embodiments. It is intended that the following claims define the scope of the present disclosure and that structures and methods within the scope of these claims and their equivalents be covered thereby.

1.-20. (canceled)

21. A method for determining postprandial glucose information to assess glucose intolerance of a patient, the method comprising:
receiving glucose data from sensor electronics coupled to an in vivo glucose sensor comprising a portion configured to be placed in fluid contact with a bodily fluid of the patient under a skin surface to generate signals indicative of glucose levels over an analysis period;
determining at least one meal event in the analysis period;
determining a pre-meal glucose parameter based on the glucose data collected in a first period before the at least one meal event;
determining a post-meal glucose parameter based on the glucose data in a second period following the at least one meal event;
determining a postprandial glucose metric based on the pre-meal glucose parameter and the post-meal glucose parameter; and
outputting the postprandial glucose metric to a computing device associated with a health care provider for display on the computing device, wherein the postprandial glucose metric is indicative of impaired glucose tolerance.

22. The method of claim 21, wherein determining the meal event comprises determining a meal start time.

23. The method of claim 21, wherein determining the meal event comprises receiving a user input of meal information.

24. The method of claim 21, wherein determining the meal event comprises identifying a trend of increasing glucose levels in a predetermined time of day period corresponding to a meal.

25. The method of claim 21, wherein the pre-meal glucose metric comprises a median of the glucose data in the first period and a maximum glucose level of the glucose data in the second period.

26. The method of claim 21, further comprising outputting a maximum glucose level based on the glucose data in the second period following the meal event.

27. The method of claim 21, further comprising outputting a median glucose level based on the glucose data in the second period following the meal event.

28. The method of claim 21, further comprising outputting a diagnosis of impaired glucose tolerance for display on the computing device.

29. The method of claim 21, further comprising providing an assessment of impaired glucose tolerance based on a comparison of the postprandial glucose metric to a threshold.

30. The method of claim 21, wherein the analysis period comprises a plurality of days.

31. A non-transitory computer-readable medium having instructions stored thereon that, when executed by at least one computing device, cause the at least one computing device to perform operations comprising:
receiving glucose data from sensor electronics coupled to an in vivo glucose sensor comprising a portion configured to be placed in fluid contact with a bodily fluid of a user under a skin surface to generate signals indicative of glucose levels over an analysis period;
determining at least one meal event in the analysis period;
determining a pre-meal glucose parameter based on the glucose data collected in a first predetermined period before the at least one meal event;
determining a post-meal glucose parameter based on the glucose data in a second predetermined period following the at least one meal event;
determining a postprandial glucose metric based on the pre-meal glucose parameter and the post-meal glucose parameter; and
outputting the postprandial glucose metric to a computing device associated with a health care provider for display on the computing device, wherein the postprandial glucose metric is indicative of impaired glucose tolerance.

32. The computer-readable medium of claim 31, wherein determining a meal event comprises determining a meal start time.

33. The computer-readable medium of claim 31, wherein determining a meal event comprises receiving a user input of meal information.

34. The method of claim 31, wherein determining the meal event comprises identifying a trend of increasing glucose levels in a predetermined time of day period corresponding to a meal.

35. The method of claim 31, wherein the pre-meal glucose metric comprises a median of the glucose data in the first period and a maximum glucose level of the glucose data in the second period.

36. The computer-readable medium of claim 31, further comprising outputting a maximum glucose level based on the glucose data in the second predetermined period following the meal event.

37. The computer-readable medium of claim 31, further comprising outputting a median glucose level based on the glucose data in the second predetermined period following the meal event.

38. The computer-readable medium of claim 31, further comprising outputting a diagnosis of impaired glucose tolerance for display on the computing device.

39. The computer-readable medium of claim 31, further comprising providing an assessment of impaired glucose tolerance based on comparison of the postprandial glucose metric to a threshold.

40. The computer-readable medium of claim 31, wherein the analysis period comprises a plurality of days.
